(19) United States
(12) Patent Application Publication
(10) Pub. No.: US 2010/0147312 A1
(43) Pub. Date: Jun. 17, 2010

(54) Respiratory Access Port Assembly With Pin Lock and Method of Use

(76) Inventors: John Brewer, Marietta, GA (US); Cassandra E. Morris, Roswell, GA (US); Joe Gordon, Mansfield, MA (US); Stephen J. Gianelis, Abington, MA (US); David A. Zitnick, Providence, RI (US)

Correspondence Address: KIMBERLY-CLARK WORLDWIDE, INC.;Tara Pohlkotte, 2300 Winchester Rd., NEENAH, WI, 54956, US

(21) Appl. No.: 12/642,972

(22) Filed: Dec. 21, 2009

Related U.S. Application Data
(63) Continuation-in-part of application No. 12/333,916, filed on Dec. 12, 2008, now abandoned.

(51) Int. Cl. A61M 16/08

(52) U.S. Cl. 128/207.14

(57) ABSTRACT

A respiratory access assembly includes a distal plate having a port, which is adapted to be positioned in operable communication with an artificial airway of a patient. The assembly includes a distal plate having a port and a proximal plate which has a first port and a second port. The distal plate is positioned against the proximal plate in a stacked configuration, and each plate is configured to move relative to the other. The assembly has an actuator which is positioned adjacent to at least one plate. The actuator cooperates with both plates to substantially prevent movement of the plates when the port of the distal plate is positioned in an alignment with at least one port of the proximal plate and an object, such as a suction catheter, is positioned through the aligned ports of the plates. The actuator cooperates with at least one plate to permit movement of at least one plate when (a) no object is positioned through aligned ports, and (b) when no ports are aligned. The assembly may have predetermined positions, which include a first, open position, a second, open position, and a third, closed position. In the first open position, the port of the distal plate and the first port of the proximal plate are aligned. In the second open position, the port of the distal plate and the second port of the proximal plate are aligned. In the third closed position, the port of the distal plate, and the first and second ports of the proximal plates are blocked to prevent an object from being passed therethrough.

This application is a continuation-in-part of U.S. Ser. No. 12/333,916 entitled “Rotating Respiratory Access Port Assembly with Push Button Lock and Method of Use” by John Brewer et al., filed Dec. 12, 2008, which is hereby incorporated by reference herein for all purposes.

### BACKGROUND

Respiratory patient care is a dynamically developing field in medicine, ranging in its needs from infants to the aged. The range of respiratory ailments, both temporary and permanent, to which such patients are subjected are many and varied. The range of procedures for intubated patients may include the following: ventilation, aspiration, oxygenation, sampling, visual inspection, in-line sensing, pressure monitoring, flushing, medicating and/or lavage. Most problems now attempt to address multiple needs of the patient and accommodation of multiple treatments, some to be performed at the same time. The lack of equipment to easily, efficiently, and safely accomplish the multiple therapies in the best interest of the patient has been and continues to be a concern.

In low lung capacity patients, such as premature babies and adults suffering from emphysema, for example, the removal of accumulated lung secretions is a problem. Secretion removal is accomplished via a suction catheter which is temporarily positioned via a respiratory access assembly in an artificial airway, i.e., an endotracheal tube placed in a portion of the patient's respiratory tract to provide air (oxygen and other gases) to the lungs of such patients. While this procedure sounds simple, it is fraught with difficulties, particularly when a caregiver must change devices or perform other therapeutic treatments sequentially or simultaneously. It is, of course, undesirable to deprive patients of oxygen during the secretion removal process. In fact, these difficulties may also result in the patient contracting ventilator acquired pneumonia. There is a need to address and overcome these difficulties.

### SUMMARY

In response to the difficulties and problems discussed herein, a respiratory access assembly is provided. The respiratory access assembly comprises a distal plate having a port, the port adapted to be positioned in operable communication with an artificial airway of a patient. The assembly has a proximal plate including a first port and a second port, the distal plate positioned adjacent the proximal plate in a stacked configuration, each plate configured to move relative to the other. The assembly also has an actuator positioned adjacent to at least one plate, the actuator cooperating with both plates to substantially prevent movement of both plates when the port of the distal plate is positioned in an alignment with at least one port of the proximal plate and an object is positioned through the aligned ports of the plates, the actuator cooperating with at least one plate to permit movement of at least one plate when (a) no object is positioned through aligned ports, or (b) when no ports are aligned.

### BRIEF DESCRIPTION OF THE DRAWINGS

FIG. 1 is a perspective view of one embodiment of a respiratory access assembly of the present disclosure, illustrating the assembly coupled to a respiratory manifold which is connected to an artificial airway at a distal end of the assembly (upper right of the drawing), and showing a portion of a suction catheter assembly coupled to a proximal end of the respiratory access assembly.

FIG. 2 is an cross-sectional view of the respiratory access assembly of FIG. 1.

FIG. 3 is an exploded proximal perspective view of the respiratory access assembly of FIGS. 1 and 2.

FIGS. 4A and 4B are plan views (top and bottom respectively) of a distal plate of the respiratory access assembly of FIGS. 1-3.

FIGS. 5A and 5B are plan views (top and bottom respectively) of a proximal plate of the respiratory access assembly of FIGS. 1-3.

FIG. 6A is a plan view of the proximal plate of the respiratory access assembly with the latch and the distal connector. FIG. 6B is a drawing of a latch used to couple the suction catheter chamber to the proximal plate. FIG. 6C is a plan view of the proximal plate of the respiratory access assembly showing proximal plate and distal connector without the latch attached.

FIG. 7 is an exploded side view of the proximal plate of the respiratory access assembly.

FIGS. 8A, 8B and 8C are individual views of the slitted cylinder, sleeve and collar, respectively.

FIG. 9A is a perspective view of locking mechanism of the respiratory access assembly in the unlocked position and FIG. 9B is a view in the locked position.

FIGS. 10A, 10B and 10C illustrate the alignment of the port and cuff of the distal plate with the first port and first cuff of the proximal plate, with the second port and second cuff or with neither, respectively.

FIG. 11 is a perspective view of another embodiment of the respiratory access assembly of the present disclosure, illustrating the assembly coupled to a respiratory manifold which is connected to an artificial airway at a distal end of the assembly (upper right of the drawing), and showing a portion of a suction catheter assembly coupled to a proximal end of the respiratory access assembly.

FIG. 12 is an exploded view of the pin lock assembly of FIG. 11.

FIG. 13 is a perspective view of another embodiment of the respiratory access assembly of present disclosure, illustrating the assembly coupled to a respiratory manifold which is connected to an artificial airway at a distal end of the assembly (upper right of the drawing), and showing a portion of a suction catheter assembly coupled to a proximal end of the respiratory access assembly.

FIG. 14 is a view of a second open position of the assembly with the port of the distal plate and the second port of the proximal plate in axial alignment. and

FIG. 15 is an exploded view of the pin lock assembly of FIG. 14.

FIGS. 16A, 16B and 16C are drawings of the sleeve, collar and handle, respectively, of the lock embodiments of FIGS. 14 and 15.

### DETAILED DESCRIPTION

Reference will now be made in detail to one or more embodiments of the disclosure, examples of which are illustrated in the drawings. Each example and embodiment is provided by way of explanation and is not meant as a limitation. For example, features illustrated or described as part of one embodiment may be used with another embodiment to yield still a further embodiment. It is intended that these and other modifications and variations come within the scope and spirit of the disclosure.

The present respiratory access port assembly operates in a closed ventilating system and is designed to accommodate multiple access to the respiratory system of an intubated patient without compromising the closed circuit character of the system and, importantly, without interruption of the flow of ventilating gases to the patient. Access to the closed respiratory system through one or more access sites is provided, for example to ventilate the lungs of the patient with gas or gases, to aspirate secretions from the lungs, to oxygenate the lungs to eliminate or reduce residual carbon dioxide therefrom, to visually inspect selected parts of the patient's respiratory system, to sample sputum and gases, to sense parameters such as flow rates, pressure, and/or temperature, to flush with solution(s), and to administer medication, gases, and/or lavage.

Many current designs for respiratory access port assemblies may have only one port. In these instances, the suction catheter must be removed when other tasks need to be performed, such as, for example, bronchoscopy, bronchial alveolar lavage, and so forth. Opening a closed ventilating system by removing the suction catheter on such a ventilated patient can lead to infection, as noted previously. Also, current designs of multiple access port manifolds and/or assemblies do not contain a safety lock. In certain instances, due to the lack of such a safety lock, the introduction of a suction catheter through a manifold port may result in a portion of the catheter being cut off and aspirated into the patient's lungs. This can lead to significant complications, including airway blockage, infection, and even death. Further, failure to adequately seal a respiratory access assembly may cause a compromise of positive end-expiration pressure (PEEP), which in turn may cause less than optimal ventilation that can result in collapsing alveoli in the patient's lungs. The disclosed respiratory access assembly includes features which permit multiple access without opening the closed ventilation system, and it contains a passive safety lock feature which prevents loss of any portion of the suction catheter and/or other object while it is positioned within the assembly.

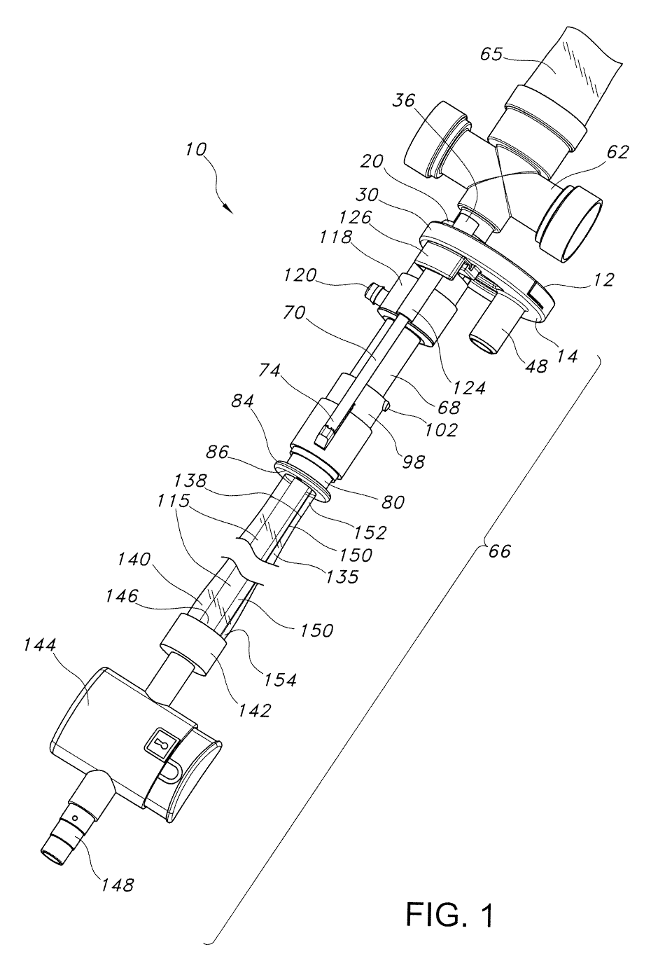

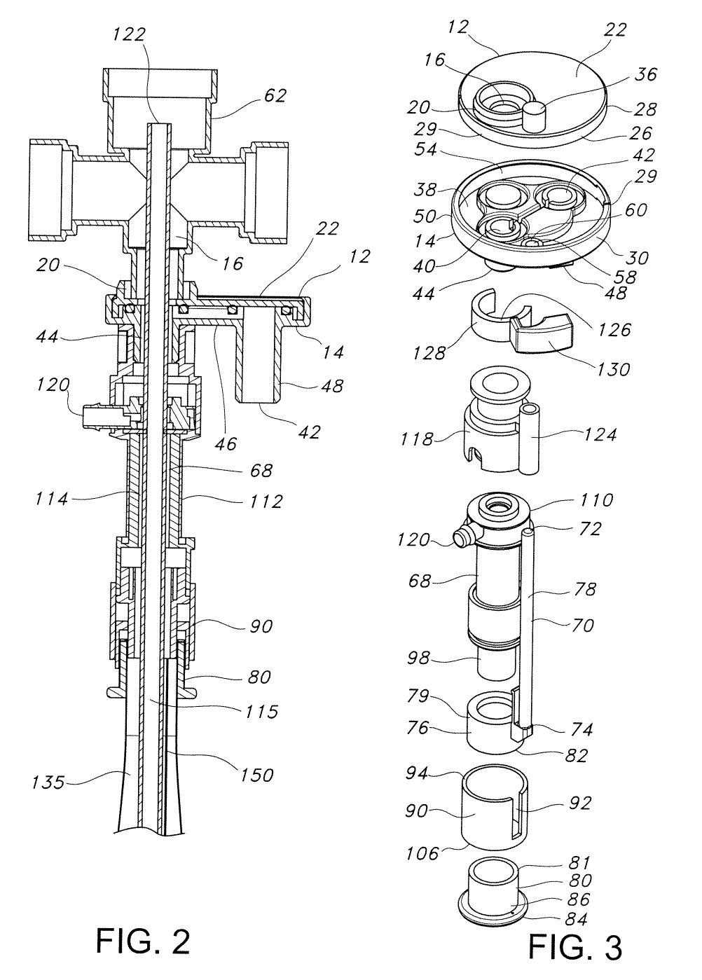

Turning now to the drawings, as illustrated in FIGS. 1 and 2, a respiratory access assembly 10 is provided. The assembly 10, as shown in FIGS. 1 and 2 includes a distal disk or plate 12 and a proximal disk or plate 14 which are positioned next to each other in a stacked and axially aligned configuration.

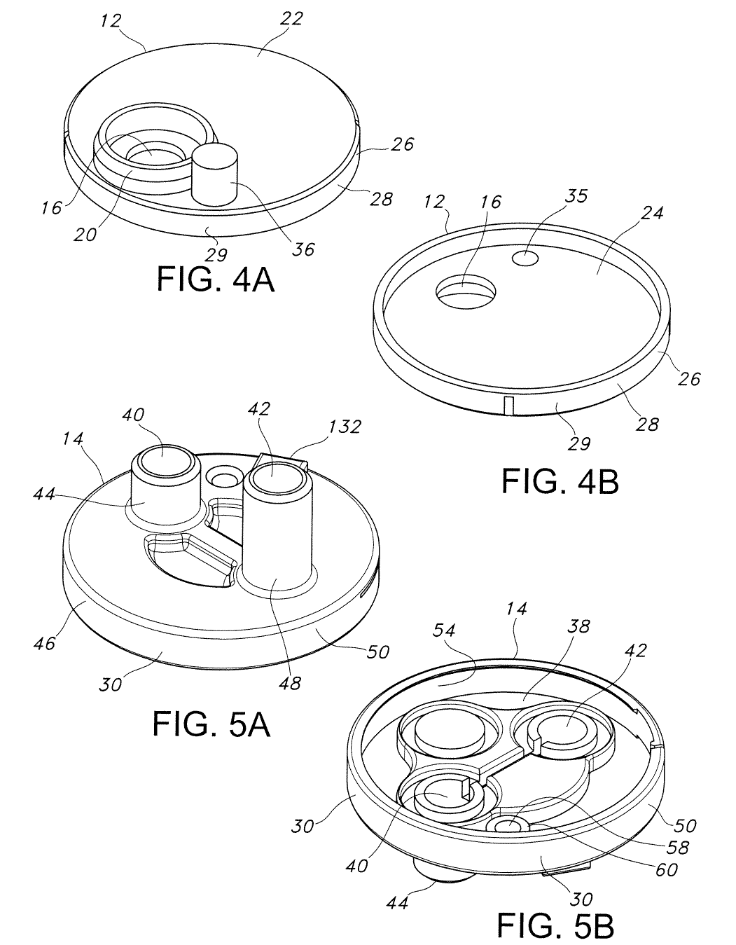

The distal disk or plate 12 includes at least one port 16 having an opening formed through the disk or plate 12, as illustrated in FIGS. 4A and 4B. A cuff, such as cuff 20, may, for example be provided on an outer distal surface 22 of the distal disk or plate 12. Such a cuff 20 generally encircles the port 16 and the opening extends therethrough, such that the cuff 20 provides a portion of the port 16. It will be understood that any cuff(s) shown and/or described herein, whether on an inner or outer surface of a disk or plate, include the characteristics and features described herein for cuff 20.

The term “port” as used herein means an opening into or through a component for the passage of an object and/or a liquid and/or a gas. The term “cuff” as used herein means a generally cylindrical component having an opening therethrough which is positioned over a port and forms a portion of the port. Further, it will be understood that a port and its cuff may collectively be given the term herein of “port”, and two or more ports, each with its associated cuff, may collectively be given the term herein of “ports”. The term “plate” as used herein refers to any shape and configuration of a plate, including, but not limited to, round, square, rectangular, and so forth. It will be appreciated that the plate may be arced, arched, planar, convex, concave, and so forth.

The distal disk or plate 12 also has a proximal surface 24 which includes an outer perimeter 26 (FIGS. 4A and 4B). A perimeter wall 28 may be formed along the outer perimeter 26. The perimeter wall 28 may extend proximally away from the outer perimeter 26 at about a 90 degree angle. The term “about”, when placed adjacent a number/numeral, refers to the stated number plus or minus ten (10) percent of the stated number. The perimeter wall 28 may also include an outer surface 29 which may include a groove (not shown) which may be configured to hold an O-ring (not shown) in the groove. Such an arrangement serves to provide at least a partial seal when it is positioned against a perimeter wall 30 of the proximal disk 14.

The proximal surface 24 of the distal plate 12 may also include a center aperture 32 configured to receive a fastener 34, such as, by way of non-limiting example, a screw or pin therethrough. The fastener 34 holds the distal and proximal plates 12, 14 adjacent to each other in a stacked and axially aligned position, while permitting movement of each plate 12, 14 relative to the other. In addition, the distal plate 12 may include an opening 35 formed in the proximal surface 24 which may extend into a closed cap 36 provided on the distal surface 22 of the distal plate 12.

The plates 12, 14 may include elements which limit their motion relative to each other. For example, one surface of one plate (i.e., for example, the proximal surface 24 of the distal plate 12) may include a semi-circular indentation (not shown). An adjacent surface of the opposite plate (i.e., for example, a distal surface 38 of the proximal plate 14) may include a tab (not shown) which sits within the semi-circular indentation (not shown). The tab and semi-circular indentation therefore cooperate to limit the movement of the distal and proximal plates 12, 14 relative to each other. In such an embodiment the rotation is desirably limited to a range of 0.1 degree to about 200 degrees. More desirably, in such an embodiment, the rotation is limited to a range of between about 1 degree and about 200 degrees.

It should be noted that a non-rotating or sliding embodiment is not illustrated herein but that such an embodiment is meant to be within the spirit of the disclosure. In such a sliding embodiment, the plates do not rotate about a common axis but slide relative to each other. In all other aspects, the embodiments are equivalent.

The proximal disk or plate 14 includes a first port 40 and a second port 42, each having an opening extending through the proximal plate 14, as shown in FIGS. 5A and 5B. The first port 40 may have a first cuff 44 which is provided on an outer proximal surface 46 of the proximal plate 14. Similarly, the second port 42 may have a second cuff 48 which is provided on the proximal surface 46 of the proximal plate 14.

The proximal plate 14 has an outer perimeter 50 which may include the perimeter wall 30. The perimeter wall 30 may be formed along the outer perimeter 50 and it extends distally away from the outer perimeter 50 at about a 90 degree angle. In the present embodiment the perimeter wall 28 of the distal plate 12 is sized to fit within the perimeter wall 30 of the proximal plate 14. In this manner, any O-ring or other sealing component on the outer surface 29 of the perimeter wall 28 of the distal plate 12 at least assists in forming a movable seal against an inner surface 54 of the perimeter wall 30 of the proximal plate 14.

A center opening 56 is provided in the proximal plate 14. The center opening 56 aligns with the center aperture 32 in the distal plate 12, and both are held at least adjacent each other by the fastener 34 (FIG. 1) positioned therethrough. In addition, an opening 58 is provided through the proximal plate 14 which may include a cuff 60 provided on a distal surface 38 thereof (FIG. 5B). It will be appreciated, however, that the opening 58 may include cuffs on either or both the proximal surface 46 and distal surface 38 of the proximal plate 14, or on neither surface 46, 38.

Turning back to the distal plate 12, in operation, the cuff 20 of the port 16 of the distal plate 12 of the assembly 10 may be coupled to a port in a manifold 62, as illustrated in FIGS. 1 and 2. The manifold 62 in turn is typically coupled to an endotracheal tube or artificial airway 64 and a ventilator (not shown). At least a portion of the artificial airway 64 is positioned in a portion of a patient's respiratory tract (not shown).

Turning now to FIGS. 7-9, a lock assembly or actuator assembly 65 is provided which, in this embodiment, provides a portion of a suction catheter assembly 66 (FIG. 1). In addition, a suction catheter chamber 68 is provided as well, which also may provide a portion of the suction catheter assembly 66. In other embodiments, it will be understood that the actuator assembly 65 may be provided separately from the suction catheter assembly 66 (not shown).

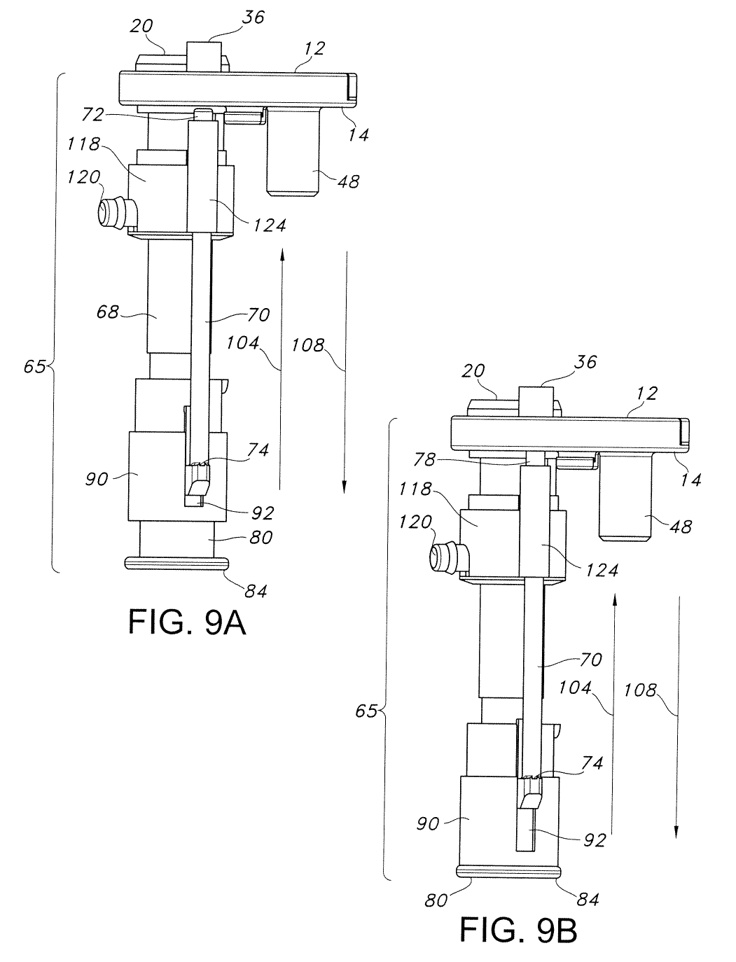

In the present embodiment, the actuator assembly 65 comprises an elongated pin 70, as shown in FIG. 7. It will be understood, however, that configurations other than a pin, rod, and so forth, may be used as well. The pin 70 includes a free distal end 72, an opposing proximal end 74 coupled to a sleeve 76. The pin 70 has an elongated body 78 positioned between ends 72, 74. The sleeve 76 may include an outer wall 79 and an inner wall 79′ (FIG. 8B). The sleeve 76 is positioned proximally in a coaxial alignment with a collar 80 (FIG. 8C). A distal end 81 of the collar 80 is positioned such that at least a portion of the collar 80 extends between the inner wall 79′ and the outer wall 79 of the sleeve 76. A proximal end 82 of the sleeve accepts movement of the collar 80 within it. At least a portion of the collar 80 may be positioned to push against the proximal end 82 of the sleeve 76, thereby moving the sleeve 76 distally. To that end, the collar 80 may include a flange 84 which extends radially outward, around the collar 80. In this embodiment, the flange 84 is positioned about a proximal end 86 of the collar 80, and may assist a health care provider in pushing the sleeve 76 distally. Movement between the collar 80 and the sleeve 76 may be limited or controlled by one or more structures provided on the collar 80 and/or the sleeve 76 (not shown). The sleeve 76 is positioned distally in a coaxial alignment with a slitted cylinder 90, which is positioned at least partially, and may be positioned substantially, over the sleeve 76. The cylinder 90 has a slit 92 which intersects a distal end 94 of the cylinder 90 (FIG. 8A). The proximal end 74 of the pin 70 is positioned to extend through the slit 92. The distal end 94 of the cylinder 90 may be coaxially aligned and positioned over a proximal end portion 98 of a suction catheter chamber 68. The proximal end portion 98 may include a radially-positioned stop tab 102 (FIG. 1) thereon, to prevent the cylinder 90 from further movement in a distal direction 104 (FIGS. 9A and 9B). Similarly, a proximal end 106 of the cylinder 90 may be coaxially positioned over at least a portion of the sleeve 76, which may be positioned over a portion of the collar 80. The movement of the cylinder 90 is limited in a proximal direction 108 (FIGS. 9A and 9B) when the proximal end 106 of the cylinder 90 abuts the flange 84 on the collar 80.

The suction catheter chamber 68, which in this embodiment may provide a portion of the suction catheter assembly 66, includes the proximal end 98 and a distal end 110 having an elongated body 112 positioned therebetween (FIG. 3). The suction catheter chamber 68 has a lumen 114 (FIG. 2) formed therethrough which is configured to permit the passage of a suction catheter 115 therethrough. A distal connector 118 is positioned on the distal end 110 of the suction catheter chamber 68. An access port 120 (FIG. 1) may be provided which intersects the lumen 114 of the suction catheter chamber 68, to permit a liquid and/or gas to be inserted and/or withdrawn. The suction catheter chamber 68 may also include one or more diaphragm valves, flapper valves, wipers, and so forth (not shown), so that a distal suction catheter tip 122 may be wiped and/or isolated therein, and air and/or a liquid cleaning solution may be provided via the access port 120 to clean the distal suction catheter tip 122. Such an air and/or liquid solution may be removed via the access port 120 and/or through a suction source (not shown) connected to a portion of the suction catheter assembly 66, as described in further detail below. Alternatively, the suction catheter 115 and the suction catheter tip 122 may be maintained in a suction catheter sheath, which will also be described in further detail below.

The distal connector 118 is configured to couple to a port, usually the first port 40 via the first cuff 44 of the proximal plate 14. The distal connector 118 includes a pin channel 124 through which at least a portion of the pin 70 may be inserted and movably held. It will be understood that the pin 70 moves through the pin channel 124 in both the distal and proximal directions 104, 108 (FIGS. 9A and 9B). The pin channel 124 directs the distal end 72 of the pin 70 through the opening 58 in the proximal plate 14 and into the opening 35 in the closed cap 36 of the distal plate 12 (FIGS. 4A and 4B and 7), so that the pin 70 secures and releasably locks the distal and proximal plates 12, 14 together in a non-movable position.

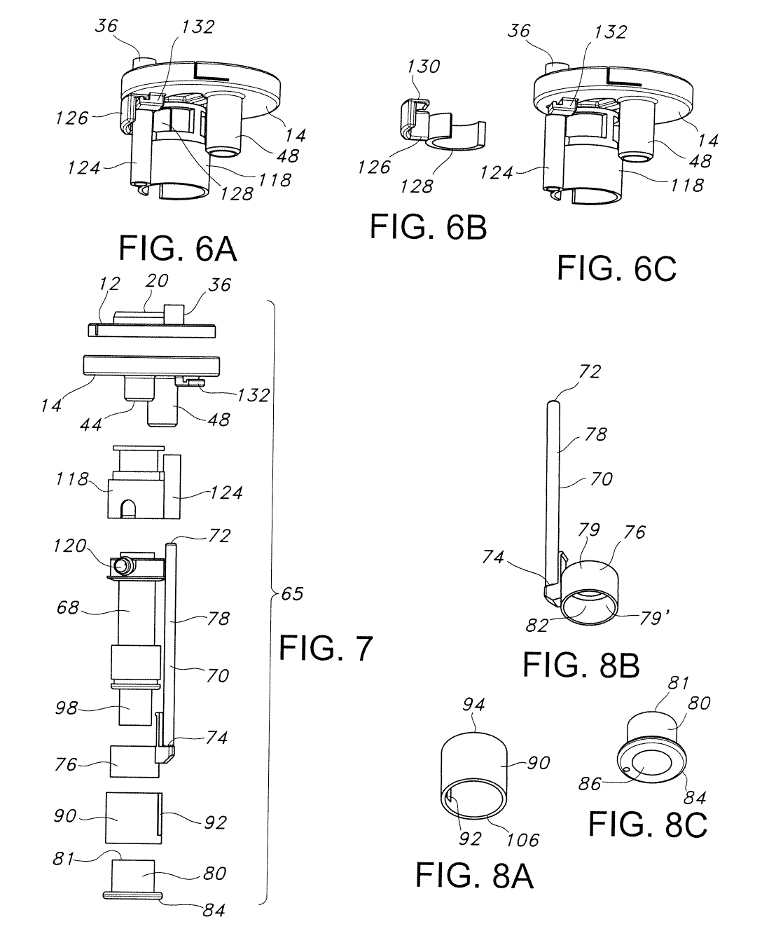

A releasable latch 126 may also be provided adjacent to the suction catheter chamber 68, and preferably next to the distal connector 118. One end 128 of the latch 126 couples to the distal connector 118. An opposing free end 130 of the latch 126 couples to a tab 132 on the proximal surface 46 of the proximal plate 14. The free end 130 of the latch 126 and the tab 132 cooperate to releasably directly couple the suction catheter chamber 68 and indirectly couple the actuator assembly 65 to the proximal plate 14 (FIGS. 6A, 6B and 6C).

The term “couple” and variations thereof, includes, but is not limited to, joining, connecting, fastening, linking, tying, adhering (via an adhesive), or associating two things integrally or interstitially together. It will be understood that two things may be coupled directly or indirectly together.

The suction catheter assembly 66 may include a sheath 135 which may be coupled at its distal end 136 to the collar 80 (FIG. 1). Alternatively, a connector (not shown) may be positioned at the distal end 136 of the sheath 134 while the connector is releasably coupled to the collar 80 (not shown).

The sheath 135 extends at least substantially over the entire length of the suction catheter 115. The terms “substantial” or “substantially” refer to something which is done to a great extent or degree; a significant or great amount; for example, as used herein “substantially” as applied to “substantially covered” means that a thing is at least 70% covered.

The sheath 135 is coupled at its proximal end 140 to a proximal end connector 142, which may include a valve or controller 144 for controlling the amount of suction pressure which is applied to the suction catheter 115. A proximal end 146 of the suction catheter 115 may be coupled to the controller 144. In addition, a connector 148 may be coupled to a portion of the suction catheter 115 and/or the proximal end connector 142 or the controller 144. The connector 148 is configured to couple to a suction source (not shown).

The suction catheter 115, when not in use, is held substantially in the sheath 135. The suction catheter distal tip 122 (FIG. 2) includes at least one opening therein (not shown). The suction catheter 115 also includes an elongated body having a lumen formed therethrough and an open proximal end (not shown). The proximal end 146 of the suction catheter 115 or, as noted previously, a portion of the suction catheter assembly 66, is adapted to couple, directly or indirectly, to at least a portion of a suctioning source (not shown) which provides a suctioning force to the suction catheter 115. It will be appreciated that the suction catheter 115 has a length which is sufficient to permit at least a distal portion of the suction catheter 115 to be positioned through the respiratory access port 10 and through any attached manifold 120 and artificial airway 122 so that it extends into a portion of a patient's respiratory tract in order to suction secretions therefrom.

When suctioning of secretions is desired, the suction catheter 115 is advanced through the aligned ports 16, 40 by the health care professional. As the catheter 115 is moved distally toward the patient, this action causes the movement of the collar 80 and associated pin 70 in the distal direction as well. The distal end 72 of the pin 70 moves through the opening 58 in the proximal plate 14 and into the opening 35 in the closed cap 36 of the distal plate 12, thus preventing movement of the plates 12, 14 relative to each other. This is the passive lock operation. Suction is then applied via the connector 148 and valve 144.

When the suction force is discontinued, it will be understood that the suction catheter 115 is then withdrawn from the patient's respiratory tract, the artificial airway 64, the manifold 62, and at least a portion of the respiratory access assembly 10, respectively. The substantial portion of the suction catheter 115 is returned to its position in the sheath 135 of the suction catheter assembly 66. Alternatively, the suction catheter 115, including the suction catheter tip 122, may be returned to the sheath 135 (not shown). It may be desirable for the distal tip 122 to remain in the suction catheter chamber 68, for reasons described previously. In this manner, the substantial length of the suction catheter 115 is contained within the sheath 135 and it is therefore positioned outside of the closed circuit ventilation system of the patient until needed again for suctioning secretions.

A lanyard or tether 150 may be provided within the sheath 135. Its distal end 152 may connect to the collar 80 and its proximal end 154 may connect to the proximal end connector 142 or other structure defined herein, so that its length extends between the collar 80 and the proximal end connector 142. The tether 150 keeps the sheath 135 from being overly extended, over-withdrawn or over-stretched. It maintains the correct length of the sheath 135 for holding the substantial portion of the suction catheter 115. The tether 150 also prevents the sheath 135 from tearing due to overstretching, thereby maintaining the integrity and operability of the sheath 135 for both holding the suction catheter 115 without exposure of the catheter 115 via a tear in the sheath 135, and holding the suction catheter 115 at a correct length within the sheath 135. Since a proximal end 146 of the suction catheter 115 may also be coupled to the proximal end connector 142 or the controller 144 associated therewith, it may be easy for a health care provider to slowly pull the proximal end connector 142 or controller 144 in a proximal direction 108 away from a patient to withdraw the suction catheter 115 from a suctioning or operative position. In this manner, the suction catheter 115 may be returned or positioned in a non-suctioning or non-operative position in the sheath 135, with a portion of the distal tip 122 of the suction catheter 115 held in the suction catheter chamber 68. An exemplary tether may be found in U.S. patent application Ser. No. 12/562,223 filed Sep. 18, 2009.

In addition, the action of the health care provider in pulling the proximal end connector 142 in a proximal direction 108 also moves the collar 80 in the proximal direction 108 because of force transmitted through the tether 150. This action moves the suction catheter 115 into the sheath 135, while also un-locking the distal and proximal plates 12, 14. That is, the action also moves the pin 70 proximally out of its locked position in the opening 35 in the closed cap 36 of the distal plate 12, through the distal and proximal plates 12, 14, and into an un-locked position relative to the distal and proximal plates 12, 14, therefore permitting movement of the plates 12, 14. This is a passive un-locking operation. The tether 150 provides the strength and durability to permit the action of moving the substantial portion of the suction catheter 115 back into the sheath 135, while the sheath 135 is spared from the force of the action and prevented from stretching or tearing.

Suction catheters are well known and widely commercially available for many medical uses. Suctioning may be performed using an “open” or “closed” system. In the open system, the suction catheter is merely a flexible plastic tube that is inserted into the flexible lumen with a source of suction connected to the proximal end of the suction catheter. Anything that the suction catheter touches before entering the lumen must be maintained in a sterile condition so a “sterile field” must be created on or next to the patient. The suction catheter must be carefully handled after it is used since it will be coated with the patient's secretions. In contrast, in the “closed” system, for example that disclosed in U.S. Pat. No. 4,569,344, a device which may be used to suction secretions is enclosed within a generally cylindrical plastic bag to eliminate or minimize contamination of the suction catheter prior to use. This is generally referred to as a “closed suction catheter” and is available under the tradename TRACH CARE® from BALLARD® Medical Products or KIMVENT® (both from Kimberly-Clark Corporation). Closed suction systems are generally preferred by healthcare providers because they are less likely to spread infection to the patient and the healthcare provider. The present respiratory access port assembly 10 may be used with such a closed suction assembly.

It will be understood that the distal connector 118 of the suction catheter assembly 66 may be coupled to either the first or the second cuff 44, 48 of the first or second ports 40, 42 of the proximal plate 14, respectively. Similarly, a bronchoscope, bronchoalveolar (BAL) catheters (e.g. BAL CATH® from Ballard Medical Products Inc., (Kimberly-Clark Corporation) or other instrumentation, and so forth (not shown), may be releasably coupled to one of the first or second cuffs 44, 48 of the first or second ports 40, 42, respectively, as well. In this manner, both the suction catheter assembly and another instrument may be simultaneously coupled to the respiratory access port 10. It will be further appreciated that the suction catheter 115, the suction catheter assembly 66, and any instruments are maintained as a part of the closed circuit ventilation system at all times.

In a method of use or operation, a health care provider may position the assembly 10 in three pre-selected positions for use. In a first position, a health care provider grasps the manifold 62 which is coupled to the cuff 20 of the port 16 of the distal plate 12. The health care provider also grasps the perimeter wall 30 of the proximal plate 14 or the first and/or second cuffs 44, 48 of the proximal plate 14 and rotates the proximal plate 14 relative to the distal plate 12 such that the first cuff 44 and first port 40 of the proximal plate 14 aligns with the cuff 20 and the port 16 of the distal plate 12.

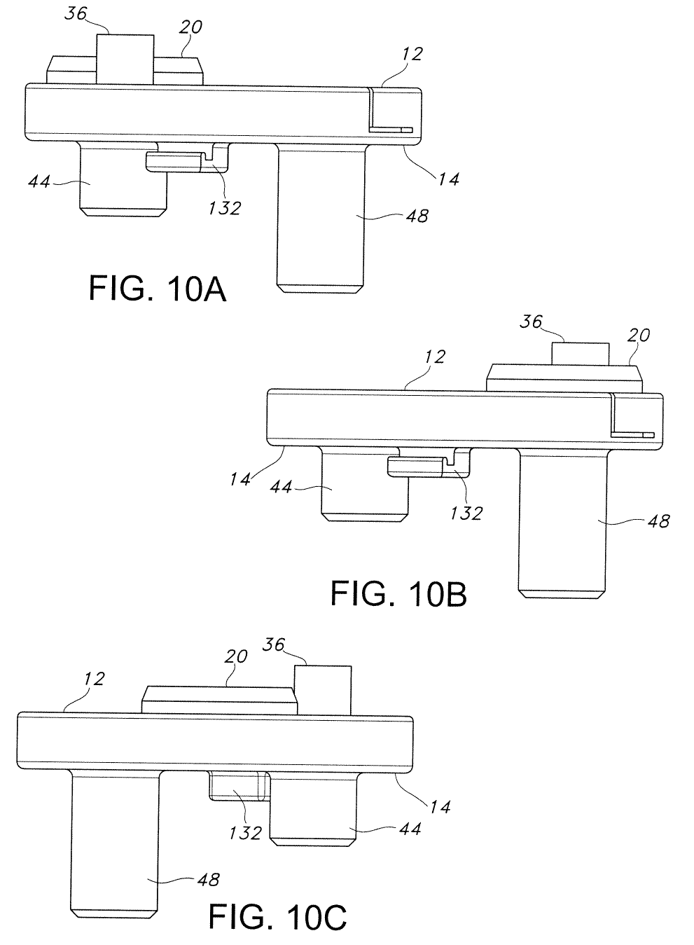

Therefore, a health care provider may grasp a portion of the proximal plate 14 and rotate the proximal plate 14 in a counterclockwise or one direction in order to permit alignment of the port 16 and cuff 20 of the distal plate 12 with the first port 40 and first cuff 44 of the proximal plate 14, as illustrated in FIG. 10A, with the second port 42 and second cuff 48 (FIG. 10B) or with neither (FIG. 10C). Alternatively, it will be understood that the proximal plate 14 may be held by a health care provider while the distal plate 12 may be moved in a clockwise or another direction. However, the distal plate 12 and its port 16 and cuff 20 are coupled to the manifold 120, which is coupled to a patient's artificial airway 122, all of which is preferably maintained in a relatively fixed position. Therefore, it will be understood that it is more desirable to hold the distal plate 12 stationary while rotating the proximal plate 14.

The phrase “stationary”, “stationary plate” and/or “stationary disk” refers to either the proximal plate or the distal plate when that plate, or component holding that plate, is grasped by a health care provider and held in a relatively fixed “stationary” position while the opposite plate or disk is moved or rotated to one of the three pre-determined positions by a health care provider. Both plates may be relatively “stationary plates” as well when the plates are positioned and locked together in a fixed, unmoving position.

The phrase “rotating plate” and/or “rotating disk” refers to either the proximal plate or the distal plate when the plates are unlocked, so that each may move or rotate relative to the other. The distal and proximal plates are configured to be positioned in three pre-determined positions. When un-locked, however, both distal and proximal plates are free to move or rotate relative to each other, and each plate may move or rotate in a direction opposite (preferably up to about 200 degrees or less) relative to each other. Both plates may move or “rotate” as well when the plates are positioned in the un-locked position so that each plate is free to be rotated in opposite directions by a health care provider.

When the ports 16, 40 and cuffs 20, 42 of the distal and proximal plates 12, 14, respectively, are in an alignment, they may be releasably held, but not locked, in this specific, predetermined position by certain components, such as, for example the cooperation of the pair of tabs (not shown) on the distal surface of the proximal plate 14 and two of a plurality of ramped indentations (not shown) on the proximal surface of the distal plate 12. That is, each of the pair of tabs on the distal surface of the proximal plate move into one of the plurality of ramped indentations on the proximal plate 14. Therefore, these components cooperate to releasably hold the distal and proximal plates 12, 14 in a specific, predetermined position, such as, for example a first open position, as illustrated in FIG. 10A. In the first open position the ports 16, 40 and cuffs 20, 44, respectively, are in an axially aligned position, and are in operable communication. The phrase “operable communication” refers to a transmission or passage between two points and/or two structures for a specific purpose. In this example, operable communication would be a passage which permits gasses and/or liquid(s) to pass, and may also be configured to permit objects to pass. In addition, the term and phrase “open,” “opened” and “open position” and variations thereof, refers to a position of the aligned ports described herein to permit an object, such as a suction catheter, a portion of a bronchoscope, and so forth, move through the aligned ports and into a portion of a patient's respiratory tract.

The health care provider then grasps a portion of the suction catheter 115 at a point about one (1) to three (3) inches proximally from the collar 80. The health care provider then pushes the grasped portion of the suction catheter 115 distally, until his/her hand pushes against the collar 80, and the grasped portion is pushed into the assembly 10. This action is then repeated, until the desired amount of the suction catheter 115 is threaded through the assembly 10, the manifold 62, and the artificial airway 64, and into a patient's respiratory tract (not shown), to permit suctioning thereof.

When the health care provide grasps the suction catheter 115 and moves it distally through the assembly 10 and pushes the collar 80, the locking assembly or actuator assembly 65 is activated by pushing the collar 80 (FIGS. 9A and 9B). The collar 80, in turn, pushes the sleeve 76 in which the pin 70 is coupled. The sleeve 76 pushes the cylinder 90, and a portion of the pin 70 moves distally through the slit 92. The collar 80, the sleeve 76 and the cylinder 90 move distally in the distal direction 104 until the sleeve 76 and cylinder 90 are stopped by the stop tab 102 on the proximal end portion 98 of the suction catheter chamber 68.

Simultaneously with the movement of the collar 80, the free end of the pin 70 and a portion of the body of the pin 78 moves distally, in the distal direction 104 through the pin channel 124 of the distal connector 118, which directs the distal end 72 of the pin 70 through the opening 58 in the proximal plate 14 and into the opening 35 in the closed cap 36 of the distal plate 12, so that the pin 70 secures and releasably locks the distal and proximal plates 12, 14 together in the non-movable locked position (FIG. 9B).

To withdraw the suction catheter 115 from a patient's respiratory tract, a health care provider may grasp a portion of the assembly with one hand to keep it stationary, while also grasping the proximal end 146 of the suction catheter 115 or the proximal end connector 142 or the controller 144 associated therewith. The health care provider slowly pulls the proximal end connector 142 or controller 144 in a proximal direction 108, away from the patient to withdraw the suction catheter 115 from a suctioning or operative position, thereby returning the substantial portion of the suction catheter 115 to its non-suctioning or non-operative position in the sheath 135, while a portion of its distal tip 122 is held in the suction catheter chamber 68.

This action of the health care provider also moves the collar 80 in the proximal direction 108 via the tether 150. The collar 80 moves the sleeve 76, the cylinder 90, and most importantly, the pin 70 in a proximal direction 108. Therefore, the distal end 72 of the pin 70 moves proximally away from the opening 35 in the closed cap 36 of the distal plate 12 and the opening 58 in the proximal plate 14, thereby un-locking the distal and proximal plates 12, 14 from the locked position, so that they are again movable relative to each other. This is a passive un-locking operation.

When the pin 70 is positioned through the distal and proximal plates 12, 14, when the suction catheter 115 is deployed or positioned in a suctioning position within a patient's respiratory tract, the plates are locked and prevented from moving, to prevent an inadvertent cutting or guillotining of a portion of the suction catheter 115. When the suction catheter is withdrawn and the pin 70 is withdrawn from its position through the distal and proximal plates 12, 14, the plates 12 14, are movable to, for example, a second position. The terms “orientation” and/or “position” used interchangeably herein refer to the spatial property of a place or way in which something is situated; for example, “the position of the hands on the clock.”

In the second position, a bronchoscope or other instrument is connected to the second cuff 48 of the second port 42 of the proximal plate 14. The assembly 10 is moved as described previously, i.e., the proximal plate 14 is moved or rotated relative to the distal plate 12, so that the second port 42 and second cuff 48 are aligned with the port 16 and cuff 20 of the distal plate 12. The bronchoscope or other object may then be introduced into the assembly 10, the manifold 62, the artificial airway 64 and into the patient's respiratory tract.

The first position with alignment of port 16 and cuff 20 with the first port 40 and first cuff 44 may be positioned, for example about 180 degrees apart from the second position, with alignment of port 16 and cuff 20 with second port 42 and second cuff 48 (FIGS. 5A and 5B). When the assembly 10 is positioned in the first position, the second port 42 and second cuff 48 are blocked or closed by a portion of the distal plate 12. Similarly, when the assembly 10 is position in the second position, the first port 40 and first cuff 44 are also blocked or closed by a portion of the distal plate 12. Such blocking or closing cooperates with other components, such as caps 114, and so forth, to maintain PEEP pressure and to prevent confusion over which port is open by preventing introduction of an object, such as a suction catheter, bronchoscope, and so forth, into the blocked port.

The term and/or phrase “closed” or “closed position” and variations thereof, refers to a position of one or more ports in which the port(s) are not aligned, so that no large object, such as a suction catheter, a portion of a bronchoscope, and so forth, may move through the referenced “closed” port(s). A port may be “closed” or “blocked” such that an object, such as those referenced previously, are blocked or prevented from moving through the port(s). The port may not be totally blocked or closed, however, and gases and/or liquid may, in at least some instances, continued to move through a blocked or closed port.

In an optional third position, the port 16 of the distal plate 12 and the first and second ports 40, 42 of the proximal plate 14 are un-aligned, and therefore are in a “blocked” or closed position such that no instrument, catheter, and so forth, may be positioned through the ports 16, 40, 42 (FIG. 10C). That is, the distal plate 12 is moved so that its port 16 is positioned between the first and second ports 40, 42 of the proximal plate 14, thereby blocking or closing the port 16 via the proximal plate 14. Similarly, and simultaneously, the first and second ports 40, 42 of the proximal plate 14 are also blocked or closed by the distal plate 12. This third position provides a closed position for all ports. The health care provider may choose to move the plates 12, 14 into the third closed position to assist in maintaining PEEP when the ports 16, 40, and 42 will not be used for a period of time.

No locking position is provided for the second and third position relative to the actuator assembly or lock assembly 65. It will be understood, however, that additional openings or apertures in the distal and proximal plates 12, 14 may be provided to provide a locking ability in the second and third positions (not shown). In this alternative, a health care provider manually moves the collar 80 in the distal direction 104 so that the distal end 72 and a portion of the body 78 of the pin 70 is positioned through such openings or apertures to lock the distal and proximal plates 12, 14. Withdrawal of the pin 70 and unlocking of the plates 12, 14 are accomplished by a health care provider grasping and moving the collar 80 in a proximal direction 108 to withdraw the portion of the pin 70 from the distal and proximal plates 12, 14, thereby un-locking them and permitting their movement or rotation relative to each other.

Certain components herein have been described and shown at certain angles. However, it will be understood that any component may be positioned at any angle or any combination of angles, so long as the assembly operates as shown and/or described herein.

It will also be understood that curved or arched plates, convex or concave disks or plates, or flat or planar disks or plates may be used herein. Further, the disks or plates may comprise any configuration, so long as they operate as shown and/or described herein. Similarly, the disks or plates may move in varying ways, that is, the disks or plates may rotate, pivot, slide, and move in any manner, and so forth, relative to each other, so long as they operate to achieve the result(s) as shown and/or described herein. A pin, such as pin 70, may also be a blade, a square rod, and so forth.

If the distal and proximal plates are flat, square or rectangular plates (not shown), it will be appreciated that the distal and proximal plates may be positioned to slide relative to each other. In such an embodiment the actuator assembly or locking assembly 65 may be configured differently to accommodate sliding or other movement. More than one stop may be provided to limit the movement of the plates relative to each other. One skilled in the art will understand the modifications which will be required to implement this and other alternative embodiments.

The assembly 10 may include more than one port and cuff on the distal disk or plate, and more than two ports and cuffs on the proximal disk or plate (not shown). In addition, the assembly 10 may include the manifold 62, or any other manifold known in the art. Further, the assembly 10 may include a suction catheter assembly 115, or any other suction catheter assembly known in the art. In a further alternative, the assembly 10 may include both a manifold as well as a suction catheter assembly.

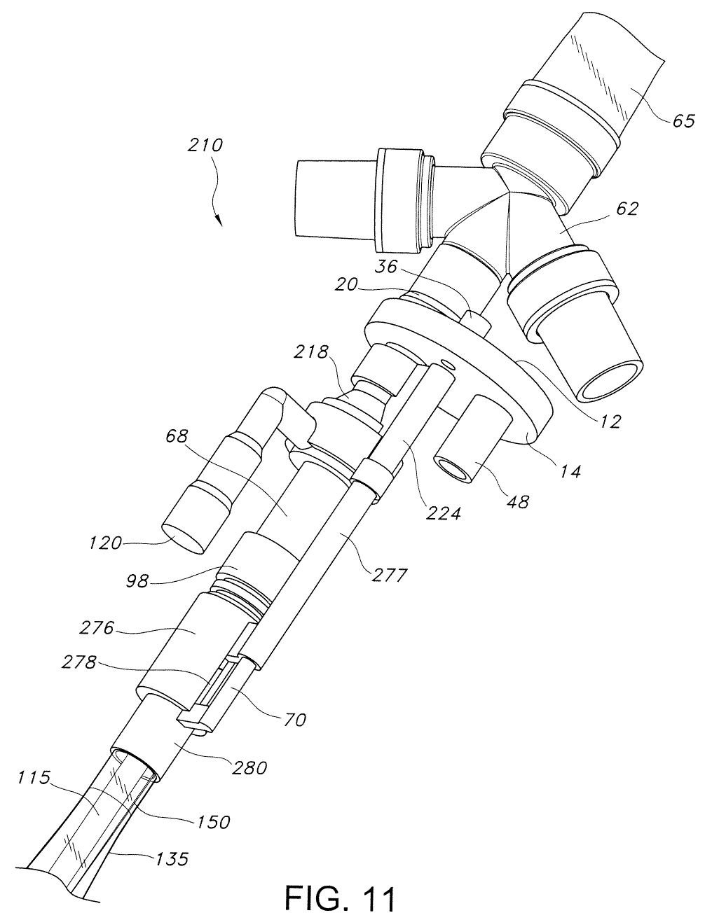

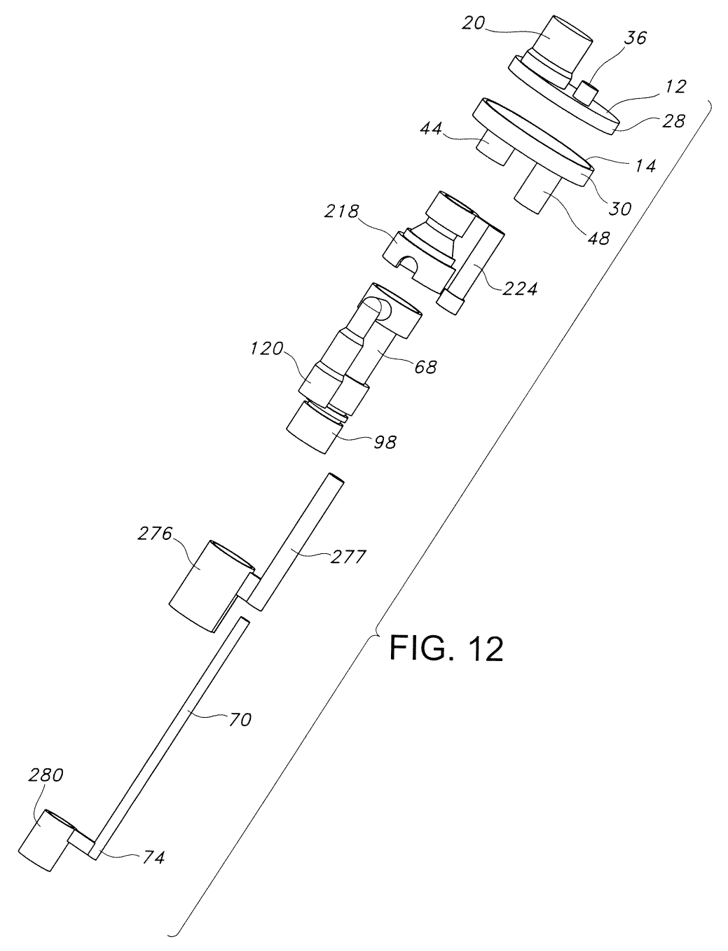

In another, similar embodiment, a respiratory access assembly 210 is provided in FIGS. 11 and 12. The respiratory access assembly 210 is similar to the respiratory access assembly 10 described herein and illustrated in FIGS. 1-9 except for a few differences. In the present embodiment, the distal end connector 218 for the suction catheter chamber 68 which includes a pin channel 224 has a different configuration. Further, there is no cylinder over the sleeve 276. The sleeve 276 includes a proximal pin cover 277 and a slit 278 below it which accepts the proximal end 74 of the pin 270. The pin 270 is coupled to the collar 280 which has no flange. The sleeve 276 and collar 280 may be coaxially aligned and coupled in such a manner which permits at least the collar 280 to move axially with respect to the sleeve 276 (not shown). There is no releasable latch provided with the present embodiment, although, optionally, one may be provided, as previously shown and described herein. The present assembly 210 may include a tether 150. Further, although not shown in this embodiment, the assembly 210 may have the same proximal end components as the assembly 10 shown in FIG. 1, and described previously in detail. It will be appreciated that the numbering of components of the present embodiment is otherwise the same as the first embodiment shown and described herein.

The assembly 210 operates in substantially the same manner as shown and/or described herein previously. It will be appreciated that the variations described above for the previous embodiment, for both configuration and operation, also apply to the present embodiment.

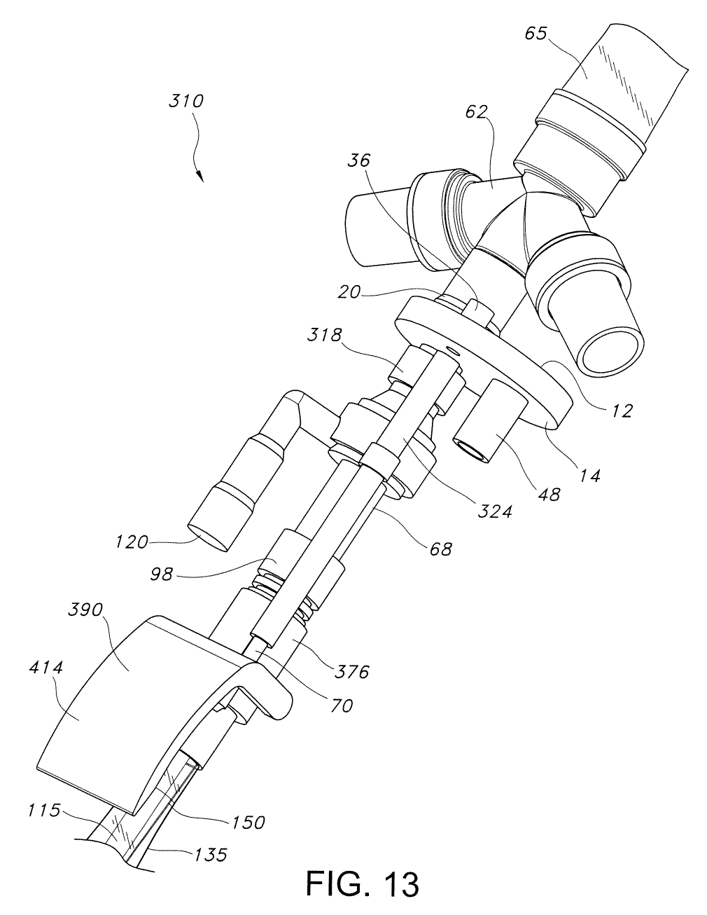

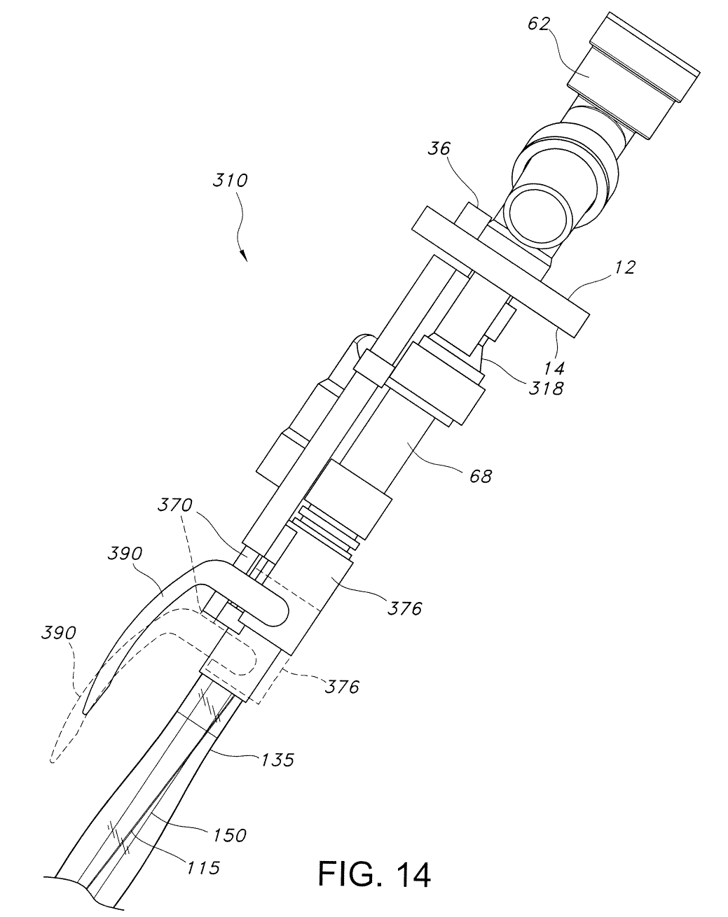

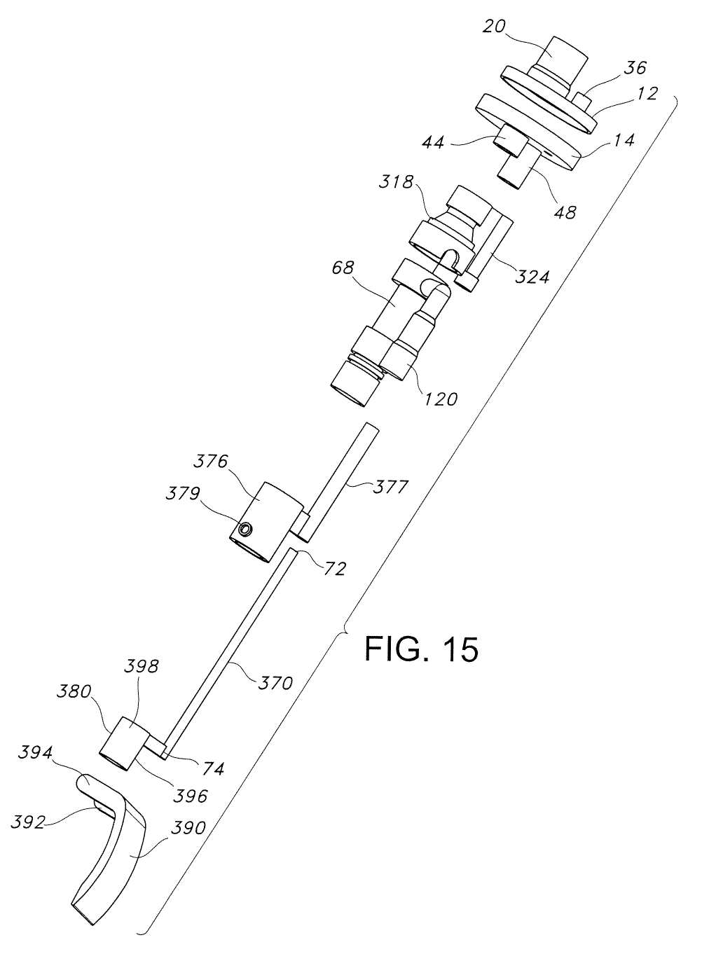

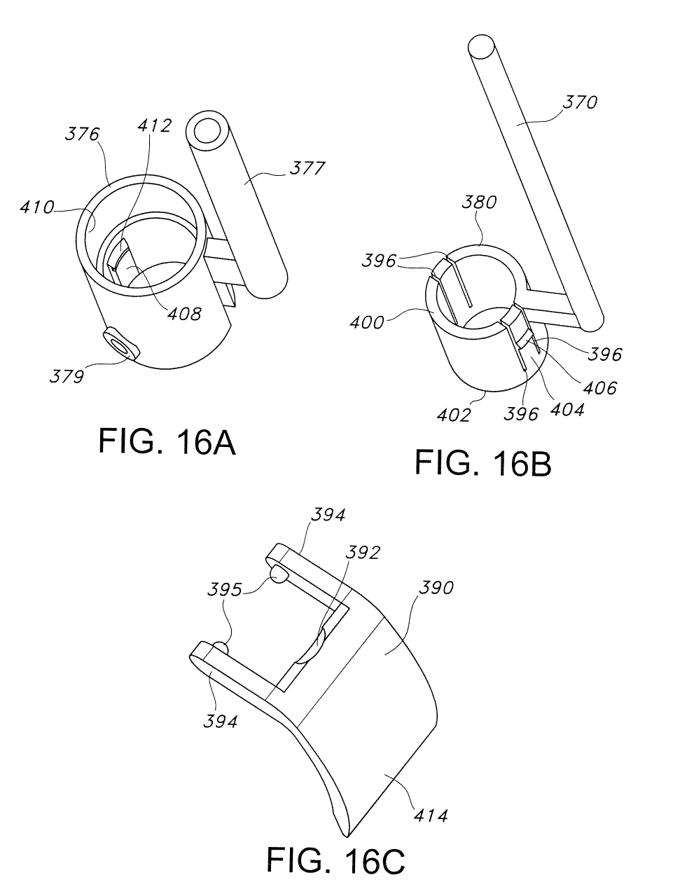

In another, similar embodiment, a respiratory access assembly 310 is provided in FIGS. 13-16. The respiratory access assembly 310 is similar to the respiratory access assembly 10 and 210 described herein and illustrated in FIGS. 1-12, except for a few differences. In the present embodiment, the distal end connector 318 for the suction catheter chamber 68 has a different configuration and a pin channel 324. Further, there is no cylinder over the sleeve 376. The sleeve 376 includes a proximal pin cover 377 and opposing circular ribs 379 positioned on the sleeve 376 at about 180 degree angle relative to each other. The sleeve 376 has a slit 378 through which a portion of the pin 370 extends. The pin 370 is coupled to the collar 380 which has no flange. The collar 380 includes a manually operable handle 390 having a ramped distal surface 392 and a pair of arms 394, one of each which has a divot or recess 395 into which one of each of the circular ribs 379 on the sleeve 376 is pivotably coupled. No releasable latch is provided with this embodiment; however, a releasable latch, as shown and described previously herein, may be utilized. The present assembly 310 may include a tether 150. Further, although not shown in this embodiment, the assembly 310 may have the same proximal end components as the assembly 10 shown in FIG. 1, and described previously in detail. It will be understood that the numbering of components of the present embodiment is otherwise the same as the first embodiment shown and described herein.

The collar 380, the pin 370 and the sleeve 376 cooperate with the handle 390. The collar 380 has a two pairs of slits 396 which extend through the wall 398 of the collar 380. Each pair of slits 396 are positioned about 180 degrees from the other. The slits are formed through the distal end 400 of the collar 380 and extend through the wall 398, ending near the proximal end 402 of the collar 380. Each pair of slits 396 forms a tongue 404 therebetween. A protuberance 406 is positioned on each tongue and extends radially outward from each tongue 404.

The sleeve 376 has a pair of grooved ramps 408 which are positioned radially about 180 degrees apart on an inner surface 410 of the sleeve 376. Each ramp 408 includes a detent 412. The ramps 408 and detents 412 thereon are configured to cooperate to slidably receive and releasably secure the collar 380 therein. That is, the collar 380 is configured to be coaxially positioned within the sleeve 376 such that the tongues 404 and the protuberances 406 thereon are moved into the ramps 408 and the protuberances 406 on the tongues 404 are releasably secured in the detents 412 in the ramps 408 of the sleeve 376. It will be appreciated that there are numerous ways to provide a connection between two or more coaxially aligned components, and the present embodiment is intended as only one non-limiting example thereof.

The handle 390 pivots on its pins 395 positioned in the circular ribs 379 of the sleeve 376 when its proximal end 414 is lifted away from the assembly 10 by a health care provider such that its distal surface 392 abuts the proximal end 374 of the pin 370. The surface 392 pushes in the distal direction 104 against the proximal end 374 of the pin 370, thereby moving the distal end 372 of the pin 370 through openings 35, 58 in the distal and proximal plates 12, 14, respectively, to lock the plates 12, 14 in a locked position. Alternatively, the handle 390 may also be used by a health care provider to slide or push the coaxially-aligned collar 380 and sleeve 376 combination in the distal direction 104 so that the distal end 372 of the pin 370 moves through the openings 35, 58 in the distal and proximal plates 12, 14 to lock the plates 12, 14 in the locked position. These two methods permit a health care provider to actively lock the plates 12, 14 together. Alternatively, the plates 12, 14 may be locked together when a health care provider's hand pushes against the handle 390 as his hand pushes the suction catheter 115 in the distal direction 104 through the assembly 310 a few centimeters at a time, thereby positioning the suction catheter tip 122 into a position in a patient's respiratory tract to permit suctioning of secretions therefrom (not shown). The suction catheter 115 may be removed from the patient's respiratory tract and moved back to its non-suctioning position by any method shown, described, taught and/or suggested herein. Some of these methods are active methods, and some are passive methods. It will be understood, however, that the distal and proximal plates 12, 14 may be unlocked when a health care provider grasps the handle 390 and pulls it in a proximal direction, thereby moving the distal end 372 of the pin 370 out of the openings 35, 58 in the distal and proximal plates 12, 14 thereby permitting the plates 12, 14 to again move relative to each other.

The assembly 310 otherwise operates in a similar manner as those shown and/or described herein previously. It will be appreciated that the variations described above for the previous embodiments, for both configuration and operation, also apply to the present embodiment, and vice versa.

While the present disclosure has been described in connection with certain preferred embodiments it is to be understood that the subject matter is not to be limited to those specific embodiments. On the contrary, it is intended for the subject matter to include all alternatives, modifications and equivalents as can be included within the spirit and scope of the following claims.

What is claimed is:

1. A respiratory access assembly, comprising:
a distal plate having a port, the port adapted to be positioned in operable communication with an artificial airway of a patient;
a proximal plate including a first port and a second port, the distal plate positioned adjacent the proximal plate in a stacked configuration, each plate configured to move relative to the other; and
an actuator positioned adjacent to at least one plate, the actuator cooperating with both plates to substantially prevent movement of both plates when the port of the distal plate is positioned in an alignment with at least one port of the proximal plate and an object is positioned through the aligned ports of the plates, the actuator cooperating with at least one plate to permit movement of at least one plate when (a) no object is positioned through aligned ports, or (b) when no ports are aligned.

2. The respiratory access assembly of claim 1, wherein the actuator is activated only when an object is positioned through the aligned ports to substantially prevent movement of the plates, thereby providing a passive lock.

3. The respiratory access assembly of claim 2 further comprising a tether that transmits forces to remove the object from the aligned ports and to permit movement of the at least one plate.

4. The respiratory access assembly of claim 3 wherein the tether is contained within a sheath that also contains a suction catheter.

5. The respiratory access assembly of claim 1, wherein when the port of the distal plate and the first port of the proximal plate are aligned, the assembly is positioned in a first open position, and the actuator is activated to substantially prevent movement of the plates when an object is positioned through the aligned ports.

6. The respiratory access assembly of claim 5, wherein when the assembly is positioned in the first position, the second port in the proximal plate is blocked by a portion of the distal plate.

7. The respiratory access assembly of claim 1, wherein when the port of the distal plate and the second port of the proximal plate are aligned, the assembly is positioned in a second open position, and the actuator is activated to substantially prevent movement of the plates when an object is positioned through the aligned ports.

8. The respiratory access assembly of claim 7, wherein when the assembly is positioned in the second open position, the first port in the proximal plate is blocked.

9. The respiratory access assembly of claim 1, wherein when the port of the distal plate is positioned between the first port and the second port of the proximal plate, no ports are aligned and the assembly is positioned in a third, closed position such that each port is blocked.

10. The respiratory access assembly of claim 1, wherein said ports are separated by about 180 degrees.

11. A respiratory access assembly, comprising:
a distal plate having a port, the port adapted to be positioned in operable communication with an artificial airway of a patient;
a proximal plate including a first port and a second port, the distal plate positioned adjacent the proximal plate in a stacked configuration, each plate configured to move relative to each other; and
a means for locking the plates together to substantially prevent movement thereof when a port of one plate is aligned with a port of another plate to provide aligned ports and an object is positioned through the ports.

12. The respiratory access assembly of claim 11, wherein the means includes an actuator positioned adjacent to at least one plate, the actuator cooperating with both plates to substantially prevent movement of the plates when the port of the distal plate is positioned in an alignment with at least one port of the proximal plate and an object is positioned through the aligned ports of the plates, the actuator cooperating with at least one plate to permit movement of at least one plate when (a) no object is positioned through aligned ports, and (b) when no ports are aligned.

13. The respiratory access assembly of claim 12, wherein the actuator is activated only when an object is positioned through the aligned ports to substantially prevent movement of the plates, thereby providing a passive lock.

14. The respiratory access assembly of claim 13 further comprising an unlocking means that removes the object from the port and deactivates the actuator, thereby passively unlock in the assembly.

15. The respiratory access assembly of claim 14, wherein the unlocking means comprises a tether

16. The respiratory access assembly of claim 12, wherein when the port of the distal plate and the first port of the proximal plate are aligned, the assembly is positioned in a first open position, and the actuator is activated to substantially prevent movement of the plates when an object is moved through the aligned ports.

17. The respiratory access assembly of claim 12, wherein when the port of the distal plate and the second port of the proximal plate are aligned, the assembly is positioned in a second open position, and the actuator is activated to substantially prevent movement of the plates when an object is positioned through the aligned ports.

18. The respiratory access assembly of claim 12, wherein when the port of the distal plate is positioned between the first port and the second port of the proximal plate, no ports are aligned and the assembly is positioned in a third closed position such that each port is blocked.
